(19) United States
(12) Patent Application Publication
(10) Pub. No.: US 2022/0331606 A1
(43) Pub. Date: Oct. 20, 2022

(54) SYSTEMS AND METHODS OF TREATING MEDICAL INDICATIONS BY ADMINISTERING DOSAGES OF LIGHT AND SOUND

(71) Applicant: INTO Technologies Inc., San Francisco, CA (US)

(72) Inventors: Stephen Auger, Santa Fe, NM (US); Jeffrey S. Bova, West Hollywood, CA (US)

(21) Appl. No.: 17/714,756

(22) Filed: Apr. 6, 2022

Related U.S. Application Data
(60) Provisional application No. 63/171,900, filed on Apr. 7, 2021.

(51) Int. Cl. A61N 5/06

(52) U.S. Cl. CPC A61N 5/0618; A61N 2005/0652; A61N 2005/0627; A61N 2005/0648

(57) ABSTRACT

A system and method for treating various medical indications is disclosed. Specific dosages of light are delivered to a subject. The dosages may be defined according to various parameters, including light wavelength, pulse frequency, intensity, area within the subject's field of vision, duration, pulse waveform shape, or a combination thereof. The dosages of light may be administered while the subject's eyes are closed. Auditory stimulus may also be provided, in synchronization with the administered dosages of light.

BY ADMINISTERING DOSAGES OF LIGHT AND SOUND

This application claims the benefit of U.S. Provisional Patent Application No. 63/171,900, filed Apr. 7, 2021, which is incorporated by reference herein in its entirety.

### CROSS-REFERENCE TO RELATED APPLICATIONS

This application incorporates by reference U.S. Provisional Patent Application No. 62/877,602, filed Jul. 23, 2019, U.S. Provisional Patent Application No. 62/961,435, filed Jan. 15, 2020, U.S. Provisional Patent Application No. 63/049,203, filed Jul. 8, 2020, U.S. Non-Provisional Patent Application No. 16/937,124, filed Jul. 23, 2020, and International Application No. PCT/US20/43324, filed Jul. 23, 2020, in their entireties.

### TECHNICAL FIELD

The present disclosure relates to systems and methods for treating medical indications (including improved human functions) by administering a dosing of light.

### BACKGROUND

Pharmaceuticals are a customary solution to treating numerous medical indications. However, there is a need for a different approach to treating these medical indications as an alternative or a supplement to pharmaceuticals.

### BRIEF SUMMARY

Disclosed are systems and methods of treating medical indications by administering dosages of light. Also disclosed are systems and methods of treating medical indications by administering dosages of light in combination with administered and synchronized auditory stimulus.

### BRIEF DESCRIPTION OF DRAWINGS

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1 illustrates a system, according to an embodiment.

FIGS. 2A and 2B illustrate examples of an arrangement of light emitters, according to an embodiment.

FIGS. 3A and 3B each illustrate an exemplary flow chart for treating a medical indication, according to an embodiment.

FIG. 4 illustrates an example computer system.

FIG. 5 illustrates an interface that may be used with the invention.

FIG. 6 illustrates an interface that may be used with the invention.

### DETAILED DESCRIPTION

The present disclosure relates to systems and methods of treating medical indications by administering dosages of light, including in combination with auditory and/or other stimuli to a subject. Exemplary devices/systems are described herein, and additional devices/systems that may be used in combination with the methods described herein are described in U.S. Provisional Patent Application No. 62/877,602, filed Jul. 23, 2019, U.S. Provisional Patent Application No. 62/961,435, filed Jan. 15, 2020, U.S. Provisional Application No. 63/049,203, filed Jul. 8, 2020, U.S. Non-Provisional Patent Application No. 16/937,124, filed Jul. 23, 2020, and International Application No. PCT/US20/43324, filed Jul. 23, 2020, each of which are hereby incorporated by reference in their entireties, and are also described herein. While specific exemplary devices/systems are described or incorporated by reference herein, it should be understood that this is done for illustration purposes only. Other components and configurations may be used without departing from the spirit and scope of the invention.

The systems and methods of the present invention may be employed to treat various medical indications by administering one or more dosage(s) of light to a user. The dosage(s) of light may be administered to the user's eyes, so as to stimulate the user's retinal ganglion cells within the user's eyes. The dosage(s) of light may be administered while the user's eyes are closed, by transmitting the light dosage(s) through the user's eyelids. The light dosage(s) may be defined according to various parameters including wavelength, area within the user's field of vision, intensity, pulse frequency, duration, pulse waveform shape, photon quantity, or any combination thereof.

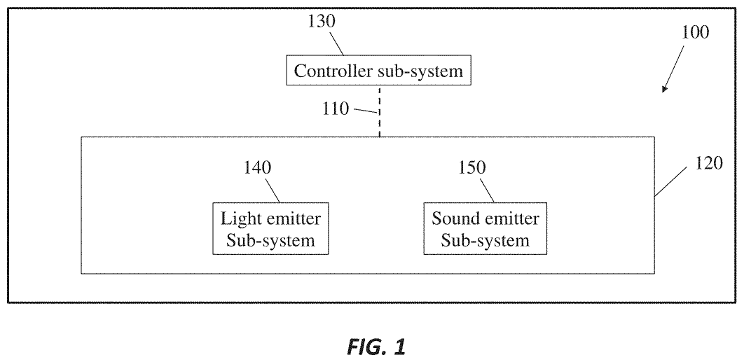

FIG. 1 illustrates a system 100 according to an example embodiment, which may be used in combination with the methods of treating medical indications disclosed herein. In one embodiment, the system 100 is configured to emit a light and/or sound stimulus to a user. According to an embodiment, the system 100 may include an emitter sub-system 120 and a controller sub-system 130. As explained in more detail below, the system 100 may be configured to apply light and/or sound stimulus to the user via the emitter sub-system 120, based on control by the controller sub-system 130.

The emitter sub-system 120 may include one or more stimulus emitters. For example, the emitter sub-system 120 may include a light emitter sub-system 140 to emit a light stimulus, and a sound emitter sub-system 150 to emit an auditory stimulus. These sub-systems as explained in detail below. In one embodiment, the light emitter sub-system 140 may be configured to apply one or more predetermined dosages of light to the user, to stimulate the user's retinal ganglion cells within the user's eyes. The predetermined dosages of light, for example, may be defined according to various parameters including:
(i) one or more predetermined wavelengths of light,
(ii) one or more predetermined areas/zones within the user's field of vision to direct the emitted light, including one or more individual areas/zones for each predetermined wavelength,
(iii) one or more predetermined intensities or amplitudes of light, including one or more individual intensities for each predetermined wavelength and/or area/zone,
(iv) one or more predetermined pulse frequencies or pulse rates (Hz), including one or more individual pulse frequencies for each predetermined wavelength, area/zone, and/or intensity,
(v) one or more durations to emit each wavelength, area/zone, intensity, and/or pulse frequency of light,
(vi) pulse waveform shape(s) (e.g., square wave, sine wave, sawtooth wave with a falling edge, sawtooth wave with a rising edge, etc.),
(vii) the quantity of photons being emitted to a user, which may depend on one or more of the above parameters, and/or
(viii) any combination of these parameters and/or other parameters.

In one embodiment, the sound emitter sub-system 150 may be configured to apply a predetermined auditory stimulus to the user. The predetermined auditory stimulus may be defined according to various parameters including:
(i) one or more predetermined beat frequencies (including any frequency and/or phase offset between the beat frequencies and the pulse frequencies emitted by the light emitter sub-system 140),
(ii) individual beat frequencies between different audio channels (e.g., left and right channels, such as to emit binaural beats),
(iii) audio frequencies of the one or more beat frequencies, including one or more individual audio frequencies for each predetermined beat frequency and/or audio channel,
(iv) one or more predetermined intensities or amplitudes of the beat frequencies, including one or more individual intensities for each predetermined beat frequency, audio channel, and/or audio frequency,
(v) one or more durations to emit each beat frequency, audio frequency, and/or intensity for each audio channel,
(vi) pulse waveform shape,
(vii) other audio emission (e.g., musical accompaniment to the beat(s)), and/or
(viii) any combination of these parameters and/or other parameters.

The controller sub-system 130 may control the emitter sub-system 120 including the light emitter sub-system 140 and/or the sound emitter sub-system 150, to control the attributes of light and/or sound stimuli to the user.

According to an embodiment, additional stimuli beyond light and auditory may be employed in the system.

Light Emitter Sub-System

The light emitter sub-system 140 may include one or more lights to deliver light-based stimulus to the user. The one or more lights may be, for example, a micro-light emitting diode (micro-LED) or LED configured to controllably emit light according to the parameters described above, based on control by the controller sub-system 130.

In one embodiment, the lights of the light emitter sub-system 140 may be configured as single-wavelength or narrow-wavelength emitters (e.g., LEDs) distributed in a predetermined arrangement so as to direct light of specific wavelength(s) (or narrow wavelength bands) to a specific field/zone of vision of the user.

In one embodiment, the light emitters are distributed in a non-uniform manner along the field/zone of vision of a user. In one embodiment, light emitters (e.g., single-wavelength emitters) emitting ultraviolet and/or purple light wavelengths are distributed with greater concentration at regions corresponding to a peripheral field/zone of vision of the user, compared to regions corresponding to a central field/zone of vision. In one embodiment, light emitters (e.g., single-wavelength emitters) emitting ultraviolet and/or purple light wavelengths are only provided at regions corresponding to a peripheral field/zone of vision of the user, and are not provided at regions corresponding to a central field/zone of vision. In one embodiment, light emitters (e.g., single-wavelength emitters) emitting red and/or infrared light wavelengths are distributed with greater concentration at regions corresponding to a central field/zone of vision of the user, compared to regions corresponding to a peripheral field/zone of vision. In one embodiment, light emitters (e.g., single-wavelength emitters) emitting red and/or infrared light wavelengths are only provided at regions corresponding to a central field/zone of vision of the user, and are not provided at regions corresponding to a peripheral field/zone of vision. In one embodiment, the light emitters are arranged in a left-right symmetrical pattern.

In one embodiment, the light emitters (e.g., single-wavelength emitters) are grouped according to their emitted wavelengths. In one embodiment, the number of groups (i.e., the number of emitted single wavelengths or narrow wavelength bands) is greater than 3. In one embodiment, the number of groups is greater than 4. In one embodiment, the number of groups is in a range between 4 and 16. In one embodiment, the number of groups is in a range between 6 and 10. In one embodiment, the number of groups is 8.

In one embodiment, the light emitters within each group may all be identical to one another. In one embodiment, at least two light emitters within an individual group may differ from one another.

In one embodiment, the groups of single-wavelength emitters correspond to respective color channels controlled by the controller sub-system 130. In one embodiment, the light emitter sub-subsystem 140 may contain 192 LEDs, split into 8 color channels, where each color channel corresponds to a different peak wavelength and has 24 identical LEDs. In one embodiment, the light emitter sub-system 140 may include 24 LEDs in each color channel, which is split into 3 smaller “pixels” of 8 identical LEDs in series. In one embodiment, the 24 “pixels” (i.e., 8 channels of 3 pixels) for each color channel are driven by a pulse width modulation (PWM) constant-current sink LED driver with an internal oscillator. The PWM driver may provide PWM control to each pixel based on a grayscale value for an individual “frame” of an experience to be provided to the user. In one embodiment, the controller sub-system 130 may provide all 3 “pixels” within a given color with the same control information (e.g., without further splitting the pixels into smaller spatial zones). Of course, it will be appreciated that the device may include a different number of color channels, a different grouping of “pixels”, a different number of total light emitters per color channel, and/or other different characteristics than those exemplary characteristics described herein.

In one embodiment, the light emitters are selected to have a narrow spectral output and to collectively summarize the visible spectrum. In one embodiment where the number of color channels is 8, the corresponding channels of wavelengths or narrow wavelength bands may be:

| | | Viewing | Luminous | |
|---|---|---|---|---|
| | Wavelength | Angle | Intensity | |
| Nominal color | (nm) | (deg) | (mcd) | Number |
| Ultraviolet | 390-395 | 130 | — | 24 |
| Purple/UV 2 | 405-410 | 130 | — | 24 |
| Blue | 468 | 130 | 115 | 24 |
| Green | 515 | 140 | 430 | 24 |
| True Green | 530 | 120 | 350 | 24 |
| Red | 639 | 130 | 54 | 24 |
| Deep Red | 660 | 140 | 16 | 24 |
| Far Red/IR | 700 | 140 | 5 | 24 |

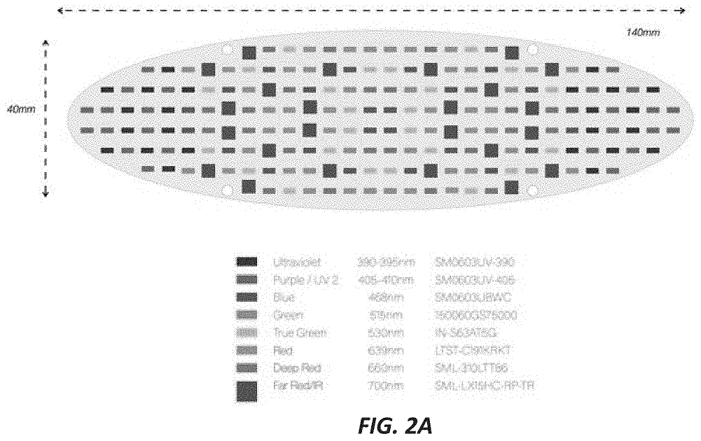

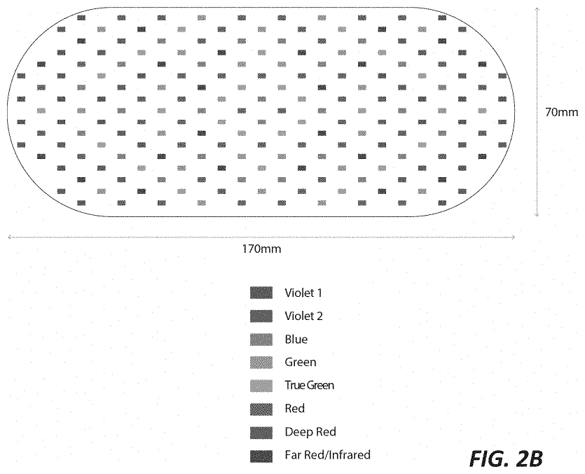

In one embodiment, single-wavelength emitters of the light emitter sub-system 140 may be arranged as illustrated in FIG. 2A.

In one embodiment, where the number of color channels is 8, the corresponding channels of wavelengths or narrow wavelength bands may be:

| | | Viewing | Luminous | |
|---|---|---|---|---|
| | Wavelength | Angle | Intensity | |
| Nominal color | (nm) | (deg) | (mcd) | Number |
| Violet 1 | 405 | 130 | — | 24 |
| Violet 2 | 415 | 150 | — | 24 |
| Blue | 468 | 130 | 115 | 24 |
| Green | 515 | 140 | 430 | 24 |
| True Green | 530 | 120 | 350 | 24 |
| Red | 639 | 130 | 54 | 24 |
| Deep Red | 660 | 140 | 16 | 24 |
| Far Red/IR | 700 | 140 | 5 | 24 |

In one embodiment, single-wavelength emitters of the light emitter sub-system 140 may be arranged as illustrated in FIG. 2B.

The arrangements of single-wavelength emitters described in FIGS. 2A and 2B may provide the capability to deliver light dosages of specific wavelengths to specific areas of a user's field of vision, while also providing a greater concentration of light emitters of particular wavelengths in certain regions (e.g., violet or ultraviolet wavelengths more concentrated at a peripheral region).

In one embodiment, at least a subset of the single wavelengths or narrow wavelength bands emitted by the light emitter sub-systems are beyond the visually perceptible range for humans. In one embodiment, the light emitter sub-system 120 and/or controller sub-system 130 are configured to emit the dosage(s) of light at least partially while the user's eyes are open. In one embodiment, the light emitter sub-system 120 and/or controller sub-system 130 are configured to emit the dosage(s) of light while the user's eyes are closed. In one embodiment, the light emitter sub-system 120 and/or controller sub-system 130 are configured to emit the dosage(s) of light only while the user's eyes are closed.

Sound Emitter Sub-System

According to an embodiment, the sound emitter sub-system 150 may include one or more speakers to deliver auditory stimulus to the user. The sound emitter sub-system 150 may alternatively or additionally include one or more interfaces (e.g., 3.5 mm audio jack, RCA or digital audio jacks, or Bluetooth) allowing the connection of peripheral audio components (e.g., headphones) for emitting the auditory stimulus to the user. For instance, wired or wireless headphones may be used for delivering binaural-beat auditory stimulus to the user. The sound emitter sub-system 150 may be configured to controllably emit audio according to the parameters described above, based on control by the controller sub-system 130.

Controller Sub-System

According to an embodiment, the controller sub-system 130 may utilize a general-purpose computing device 400, as explained in more detail below. In one embodiment, the controller sub-system 130 stores pre-programmed experiences of stimulus to present to a user, such as synchronized control sequences for the light emitter sub-system 140 and/or sound emitter sub-system 150, and controls these sub-systems accordingly to present the experience (e.g., including the defined dosing of light) to the user. In one embodiment, the controller sub-system 130 is configured to receive and store defined experiences (and/or modify existing stored experiences) based on information from an external source (e.g., over a network, from a USB storage device, based on user input and/or control parameters, etc.).

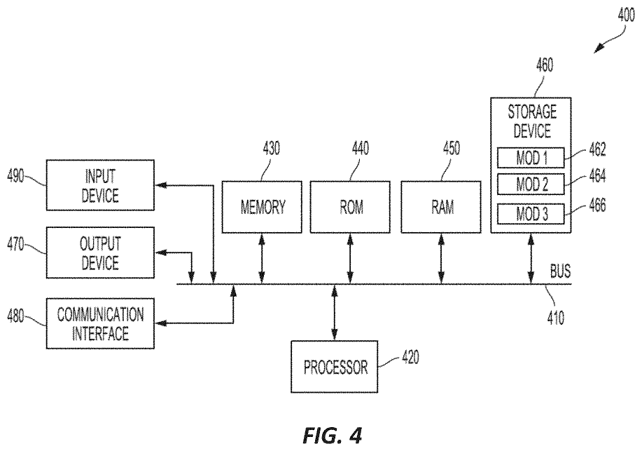

With reference to FIG. 4, an exemplary arrangement of the controller sub-system 130 described above includes a general-purpose computing device 400, including a processing unit (CPU or processor) 420 and a system bus 410 that couples various system components including the system memory 430 such as read-only memory (ROM) 440 and random access memory (RAM) 450 to the processor 420. The system 400 can include a cache of high-speed memory connected directly with, in close proximity to, or integrated as part of the processor 420. The system 400 copies data from the memory 430 and/or the storage device 460 to the cache for quick access by the processor 420. In this way, the cache provides a performance boost that avoids processor 420 delays while waiting for data. These and other modules can control or be configured to control the processor 420 to perform various actions. Other system memory 430 may be available for use as well. The memory 430 can include multiple different types of memory with different performance characteristics. It can be appreciated that the disclosure may operate on a computing device 400 with more than one processor 420 or on a group or cluster of computing devices networked together to provide greater processing capability. The processor 420 can include any general purpose processor and a hardware module or software module, such as module 1 462, module 2 464, and module 3 466 stored in storage device 460, configured to control the processor 420 as well as a special-purpose processor where software instructions are incorporated into the actual processor design. The processor 420 may essentially be a completely self-contained computing system, containing multiple cores or processors, a bus, memory controller, cache, etc. A multi-core processor may be symmetric or asymmetric.

The system bus 410 may be any of several types of bus structures including a memory bus or memory controller, a peripheral bus, and a local bus using any of a variety of bus architectures. A basic input/output (BIOS) stored in ROM 440 or the like, may provide the basic routine that helps to transfer information between elements within the computing device 400, such as during start-up. The computing device 400 further includes storage devices 460 such as a hard disk drive, a magnetic disk drive, an optical disk drive, tape drive or the like. The storage device 460 can include software modules 462, 464, 466 for controlling the processor 420. Other hardware or software modules are contemplated. The storage device 460 is connected to the system bus 410 by a drive interface. The drives and the associated computer-readable storage media provide nonvolatile storage of computer-readable instructions, data structures, program modules and other data for the computing device 400. In one aspect, a hardware module that performs a particular function includes the software component stored in a tangible computer-readable storage medium in connection with the necessary hardware components, such as the processor 420, bus 410, display 470, and so forth, to carry out the function. In another aspect, the system can use a processor and computer-readable storage medium to store instructions which, when executed by the processor, cause the processor to perform a method or other specific actions. The basic components and appropriate variations are contemplated depending on the type of device, such as whether the device 400 is a small, handheld computing device, a desktop computer, or a computer server.

Although the exemplary embodiment described herein employs the hard disk 460, other types of computer-readable media which can store data that are accessible by a computer, such as magnetic cassettes, flash memory cards, digital versatile disks, cartridges, random access memories (RAMs) 450, and read-only memory (ROM) 440, may also be used in the exemplary operating environment. Tangible computer-readable storage media, computer-readable storage devices, or computer-readable memory devices, expressly exclude media such as transitory waves, energy, carrier signals, electromagnetic waves, and signals per se.

To enable user interaction with the computing device 400, an input device 490 represents any number of input mechanisms, such as a microphone for speech, a touch-sensitive screen for gesture or graphical input, keyboard, mouse, motion input, speech and so forth. An output device 470 can also be one or more of a number of output mechanisms known to those of skill in the art. In some instances, multimodal systems enable a user to provide multiple types of input to communicate with the computing device 400. The communications interface 480 generally governs and manages the user input and system output. There is no restriction on operating on any particular hardware arrangement and therefore the basic features here may easily be substituted for improved hardware or firmware arrangements as they are developed.

Methods of Treating Medical Indications

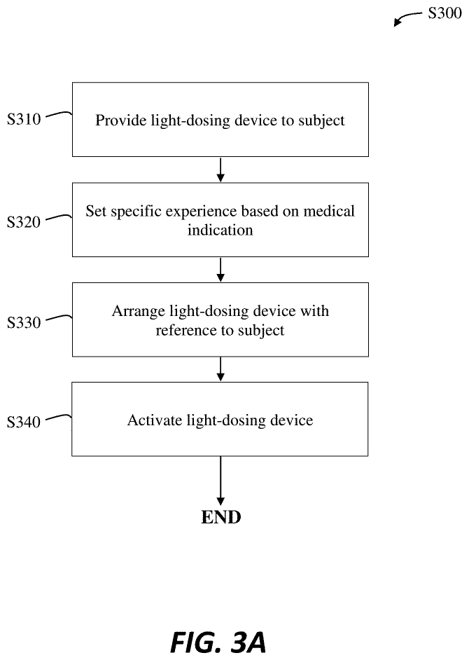

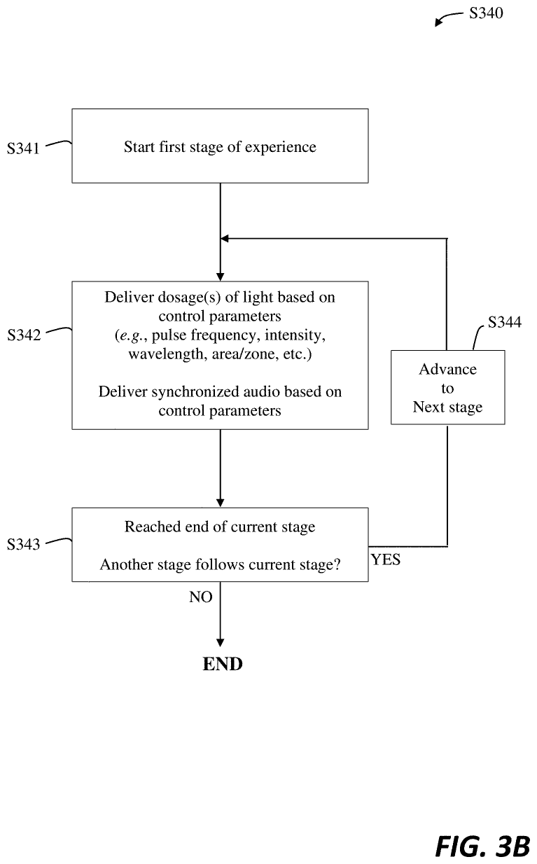

Various methods of treating medical indications according to the invention will now be described. FIG. 3A illustrates a first embodiment of a process S300 for treating medical indications in accordance with the invention. First, in step S310, the subject is provided with a light-dosing device (or system), such as those exemplary systems and devices described herein.

In step S320, a user controls the light-dosing device to set a specific light-dosing experience corresponding to a desired medical indication for treatment. For example, the light-dosing device may contain multiple programmed experiences, each corresponding to a specific medical indication for treatment. In one embodiment, the light-dosing device may contain multiple programmed experiences even for a single medical indication. The user performing the control in this step may be the subject him/herself, a medical professional, and/or any individual capable of operating the light-dosing device.

In step S330, the light-dosing device is physically arranged with reference to the subject. For instance, the device may be placed such that its light emitters face the subject's eyes. In one embodiment, the device may be physically attached to the subject. In another embodiment, the device may not be physically attached to the subject, but is retained by a retention mechanism such that it continuously faces the subject's eyes. In one embodiment, the device is placed proximate to the subject such that sound emitted from the device is perceptible by the subject. In one embodiment, peripheral audio devices (e.g., wired or wireless headphones) are physically and/or informationally coupled with the device.

In step S340, the light-dosing device is activated and administers the experience to the user. This experience may involve specific controlled and sequenced dosing of light delivered to the user's eyes based on specific control parameters, as described above. The experience may also involve specific controlled and sequenced emissions of audio information based on specific control parameters, as described above. The emissions of light and auditory stimulus may be synchronized with each other, according to the defined experience.

The experience may also involve modification of one or more parameters during the presentation of the experience. For instance, the experience may be divided into “stages”, where each successive stage involves different values of the various light and/or sound control parameters from the previous stage. As one example, the light pulse frequency may differ between different stages. As another example, the emitted light wavelengths may differ between different stages. It will be appreciated that any of the control parameters described herein may be varied between different stages. It will also be appreciated that any of the control parameters described herein may even be varied within the same stage. For instance, within a single stage, the pulse frequency may continuously alternate between two or more frequencies, may immediately change from a first frequency to a second frequency, or may ramp/sweep from a first frequency to a second frequency. The other parameters described herein may likewise vary within an individual stage and between different stages.

FIG. 3B illustrates additional steps within step S340 for presenting the experience to the user. In step S341, the controller sub-system 130 initiates the first stage of the selected experience. In step S342, the controller sub-system 130 controls the light emitter sub-system 140 to emit the defined dosage(s) of light, according to the stage of the experience. The controller sub-system 130 also controls the sound emitter sub-system 150 to emit auditory stimulus according to the stage of the experience, synchronized with the light emission.

The defined dosage(s) of light may include the definition of specific control parameters as described above, including but not limited to pulse frequency (or frequencies), intensity (or intensities), light wavelength(s), area(s)/zone(s), duration, pulse shape, and/or other control parameters. Likewise, the defined auditory stimulus may include the definition of specific control parameters as described above.

In step S343, the controller sub-system 130 determines that the end of the current stage has been reached, and determines whether another stage follows the current stage. If a subsequent stage exists in the experience, the method advances to step S344. If the current stage is the last stage in the experience, the method ends.

In step S344, the controller sub-system 130 advances to the next stage and then returns to step S342 to administer the next stage to the subject.

The inventors have discovered that the controlled dosing of light may be effective to treat various medical indications. It will be appreciated that medical indications as described herein include not only physical and/or physiological ailments, but also human function(s) to be improved (e.g., cognitive abilities, alertness, etc.). The medical indications that may be treated/improved by such controlled light dosing include, but are not limited to (organized by general category and further divided into non-limiting consumer/clinical applications where applicable):

Sleep Support

| Consumer | Clinical |
|---|---|
| Children Audio/Visual | Nighttime Urination & Rumination |
| Lullaby/Narrative | Issue Content |
| Poor Sleep & Insomnia Relief | Melatonin, DHEA, Serotonin |
| Content | Release Content |
| Bedtime Trance & Dream Content | Sub-Visual Circadian System |

Meditation/Medical

| Consumer | Clinical |
|---|---|
| Narrative Intention & Prayer | Post Stroke Stimulation Content |
| Content | |
| Mind-Body Integration, Kinesthetic | PTSD - Fear, Terror & Trauma |
| Awareness, Synesthetic Integration | Resolution Content |
| Ecstatic Trance Content | Acute Pain Reduction Content |
| Awe, Wonder, Inspiration Content | Headache & Migraine Reduction |
| Sensory Vision Quest Content | Content |
| | Pre & Post Anesthesia Support |
| | Blood Pressure (e.g., hyper- |
| | tension) & Variable Heart |
| | Rate Regulation Content |
| | Fibromyalgia Symptom Relief |
| | Content |
| | Neuropathic Pain Reduction |
| | Content (Specifically for Spinal |
| | Injury) |
| | Parkinson Symptom Relief Content |
| | Sensory Integration Support |
| | Content |
| | Autonomic Hormone & Biomarker |
| | Regulation |
| | Hybrid Pharmacological/Audio- |
| | Visual Protocols |
| | Ritlan/Adderoll/Methylphenidate |
| | Reduction Protocols |
| | Hybrid Psychiatric Protocols for |
| | Mental Disorders |
| | Pain, headache, or migrane |

Stress and Anxiety

| Consumer | Clinical |
|---|---|
| Stress & Anxiety Mitigation | Stress & Anxiety Mitigation |
| Stress Support Content for Terminal | Cortisol Reduction Content |
| Diagnosis | Content for Hospice |
| Stress Support for Chemotherapy | |
| Treatments | |
| Social & Situational Anxiety Relief | |
| Content | |
| Geriatric Boredom, Anxiety, | |
| Irritation | |
| Canine Barking Control Content | |

Relaxation

Neck & Mandibular Tension Release Content
Cannabis/Ketamine/Psychedelic Support & Enhancement Content
Open & Closed Eye Content
Primal Eco-Nature Content (Land/Ocean/Sky/Cosmos/Jungle Gaia Series)

Performance Enhancement

| Consumer | Clinical |
|---|---|
| Mood Shift & Improvement | Pre & Post Counselling, EMDR & |
| Content | Therapy Content |
| Compassion & Empathy Content | Autonomic Nervous System Self |
| Emotional Endurance & Tolerance | Regulation Content |
| Content | Dyslexia Mitigation Content |
| Irritability Improvement Content | Mood Disorder Support Content |
| Team Intention Integration & Flow | Mental Adaptation & Resilience |
| Content (Alpha Synchronization) | Content |
| Increase Working & Long-term | Cognitive Performance Content |
| Memory Content | Emotional Adaptation & Resilience |
| Creativity & Imagination | Content |
| Enhancement Content | Reactivity Reduction Content |
| Morning Wake-up Relax & Focus | Child/Adolescent Emotional |
| Content | Support Content |
| Social Bonding & Team Sync | Coping, Distress, Reactivity |
| Content | Reduction Support Content |
| Disturbance in the Field Content | Individual & Collective Grief, |
| (Rumination Reduction) | Sadness & Loss Support Content |
| Flow & Go With the Flow | |

Cognitive Intervention

ADD/ADHD/OCD/ASD Spectrum Support
Endorphin & Dopamine Release Content
Alzheimer & Dementia Support Content
Attention-deficit/hyperactivity disorder

Emotional Intervention

| Consumer | Clinical |
|---|---|
| Attachment & Separation Issues | Rage/Anger & Aggression |
| | Management Content |
| Child/Adolescent Emotional | Addiction Management |
| Support Content | Oxytocin Elevation Content |
| Post Gaming Autonomic Down- | Attachment & Separation Issues |
| shift Content | Depression Support Content |
| Coping, Distress, Reactivity | Suicidal Rumination Relief |
| Reduction Support Content | Content |
| Depression Support Content | PMS/Menopause Syndrome Relief |
| Morning Wake-up Relax & Focus | Content |
| Content | Postpartum Blues & Depression |
| Social Bonding & Team Sync | Content |
| Content | Childhood & Adult Shame & |
| Disturbance in the Field Content | Negative Self Belief Support |
| (Rumination Reduction) | Content |
| Flow & Go With the Flow | Smoking Cessation Content |
| Individual & Collective Grief, | News Cycle Addiction/Reaction |
| Sadness & Loss Support Content | Support Content |
| Childhood & Adult Shame & | Catharsis, Weeping & Crying |
| Negative Self Belief Support | Content |
| Content | Parasympathetic Nervous System |
| Autonomic Nervous System | Support Content |
| Support for VR Applications | Autonomic Nervous System |
| | Support for VR Applications |

The inventors have discovered that the light-dosing device may treat the medical indications by stimulating the user's central nervous system and metabolic systems and producing a desired brainwave state in the user.

Efficacy Study

An efficacy study of the methods described herein will now be described.

(1) Background/Motivation/Inspiration
(a) Light

The time-based light sequences that accompany the sound design incorporates 8 LED frequencies (refer to chart).

The creative composition of specific light frequencies, their intensities, and durations are informed by information and reporting related to the effect of color and light on mood. Additional research describing techniques that employ rhythmic pressure waves that emanate from drums causing subtle pulsing effects on flames, fire, candles, etc. inform the creative use of pulsing light within the compositions. These findings are garnered from cross-cultural research of indigenous cultures that utilize drumming for their ceremonies, rituals, and other activities intended to elicit meditative and dreamlike trance states.

(b) Sound

The subject when presented with rhythm, binaural beats, and light pulses, separately or in concert, tends to synchronize an aspect of themselves with one or all of these external rhythms. For example, when listening to music with a beat, one will impulsively move in synchrony to the tempo and rhythm presented.

The musical key is centered around a D drone. Drones are a key component of much music in use for meditation. Originating in East Asia and spreading in practice to the rest of the world, the drone is used for both musical effect and intentional purposes.

Advanced film scoring, electronic music, and sound design techniques are employed to create the musical atmosphere and provide an audio narrative for the listener during the experience.

These techniques are the tool kit of the modern music composer. Advanced film scoring utilizes research in the cultural, historical, and psychological context of the music required. The effect of genre, tempo, key, tonality, and rhythm to set the feeling or atmosphere, enhances, and further deepens the listener's experience.

Electronic music and sound design work together to underscore specific moods and immerse the viewer into the experience. Electronic music includes the use of synthesizers, sound samplers, and Digital audio processing (DSP). The sound designer creates custom sounds from both musical and non-musical sources to add further depth to the sound experience.

As music listening and understanding is an intricate psychological phenomenon, individual differences in perception of Musical style and genre preferences add a layer of complexity to a study like this. Any organized sound (Music) or associative sound (Nature) engages more areas of the brain. We tend to visualize imagery and have emotional responses when we associate sounds with our experience and memory of them.

Therefore the same audio material is used for the placebo, minus the binaural beats. Subjects in both groups listen to the identical music program, but the placebo group does not have the binaural rhythms to synchronize to.

The soundtrack is composed to support the user's journey through a musical form that allows the perception of different rhythmic elements.

(2) Objectives

Guided by scientific methodologies, the inventors are developing hardware and software aimed at assisting individuals with transitioning into more desirable states of consciousness, utilizing scientific results to dictate both development and forward-facing marketing materials. As such, the objective of the current study is to determine the impact of the inventor's light-dosing device on a subject's mood states as a function of different experience lengths (5.5 min, 11 min, 22 min). The impact will be compared to a placebo-matched control in a double-blind experiment whereby the placebo does not contain crucial light and sound elements critical to the presentation emitted by light-dosing device. A separate control where subjects will be guided through a breathwork meditation will provide a benchmark for impacts on mood states without the use of the device.

Success of the objective will be met if:
1. The research concludes, definitively, that inventors' device is capable of inducing meaningful changes in mood states.
2. The research identifies a dose-response curve that can be used to instruct consumers on daily use patterns most conducive to success.
3. The proceedings of this research are published in a peer-reviewed publication with an impact factor>5.
4. The proceedings permit the use of marketing claims that the scientific team feels confident in making

(3) Device Overview

This protocol makes use of a novel visual experience device developed by the inventors. The device has 192 LEDs each capable of emitting one of eight different colors. Each LED can be programmed to flash at a specific frequency for a specific duration. The visual experience is paired with a proprietary auditory composition centered around a D drone that leverages a convolution reverb simulating the intersection of a temple and a cathedral. The convolution reverb of a modeled acoustic space provides complex spatial cues to give the listener the impression of being immersed in an actual physical space; the audio master is recorded at 24 bit/48 k to provide a high-resolution listening experience capable of relaying the spatial information. The atmospheric soundscape is complemented by occasional sounds associated with meditation, including tanpura, gongs, and bells. Embedded within the audio are Binaural Beats using the inventor's proprietary application to tune to the harmonic progression of the music while simultaneously beating at designated frequencies. The device facilitates the synchronization of different rhythms within the audio to match the light pulsations. The audiovisual experience is controlled using a proprietary software plugin.

(4) Setup

In this experiment, the device will be mounted to the edge of a desk using a boom mic arm that positions the device 3 inches in front of the participant's eyes as they sit in a recliner next to the table. The device will be wired to a computer on the desk. Participants will have their eyes closed for the duration of the experience. Participants will also insert wired earbuds hooked up to the audio jack of the computer.

Participants will be fitted with a 64-channel EEG cap, using high-chloride abrasive electrolyte gel to achieve desirable impedance levels at each electrode Amplifiers will be located on a rolling cart positioned beside the recliner and data will be collected and coordinated with the peripheral recordings. All participants will also be fitted with the following peripherals: Electromyography, Bio-Impedance-Based Respiration Rate, Heart Rate Variability, Oxygen Saturation, and Galvanic Skin Response.

(5) Conditions

| | Number of |
|---|---|
| Group | Subjects |
| Experimental: Actual-Composition (10 minutes) | 32 |
| Placebo: Sham-Composition (10 minutes) | 32 |
| Control: Breath-Counting (10 minutes) | 32 |
| Experimental: Actual-Composition (15 minutes) | 32 |
| Placebo: Sham-Composition (15 minutes) | 32 |
| Control: Breath-Counting (15 minutes) | 32 |
| Experimental: Actual-Composition (20 minutes) | 32 |
| Placebo: Sham-Composition (20 minutes) | 32 |
| Control: Breath-Counting (20 minutes) | 32 |

Note:
Power analysis was conducted to determine a 20% reduction in anxiety as compared to the average STAI score from this study. Used this calculator at p = 0.05 and power = 0.80.

In all conditions, participants will receive a battery of mood assessments (see below) both before, after, and 1 week following their participation.

The experimental condition is designed to encourage a state of relaxation and includes a light composition and the atmospheric auditory composition complimented with binaural beats. The placebo condition is an asynchronous series of pulsing light frequencies that modulate the 8 LED frequencies at irregular intervals. The intensity of the asynchronous light is identical to the light levels in the study protocol, therefore matching the lux output of the study protocol. The placebo audio is identical to the Experimental condition with the exception of the Binaural beat rhythms which are absent from the audio.

Another control condition will include a simple breath-focused, eyes-closed meditation exercise, matched in duration to the experimental and placebo conditions.

Participants in the experimental/placebo conditions will receive an audiovisual experience of either 5.5, 11, or 22 minutes in length. Each contains the same content and rhythms but a 50% reduction of running time in each iteration. The duration, light pulsation frequencies used and the binaural beat frequencies at each epoch of the experience are outlined below.

(6) Notes

Pre and Post “idle” present slow evolving waves of light and sound to “warm-up” or “cool down” the user before and after the experience.

(7) Study Procedure

Protocol flow is as follows:
Pre-Screening Scheduling of applicable subject Pre-Experience Questionnaire (35 mins) Experience (5.5, 11, or 22 mins) Post-Experiment Questionnaire (40 mins) One-Week Follow Up Questionnaire (40 mins; same as post-experiment).

The total time for this experiment will be either 120, 126, or 137 minutes depending on which group the subject is randomly assigned to.

(8) Battery of Mood Assessments (Before)

Hospital Anxiety and Depression Scale (HADS) (Zigmond & Snaith, 1983), Global Anxiety Visual Analog Scale (GA-VAS) (Williams et al., 2010), State-Trait Anxiety Inventory (STAI) (Spielberger, 2010), Profile of Mood States (POMS 2-A) (Searight, 2017)), Local-Global Task (Navon, 1977), Stroop Task (Stroop, 1935), Five Facet Mindfulness Questionnaire, NEO-FFI (Specifically the Openness to Experience aspect) (Baer et al., 2006), Phenomenological Consciousness Inventory (PhCI) (Pekala et al., 1991).

(9) Battery of Mood Assessments (After/1-Week After)

Hospital Anxiety and Depression Scale (HADS) (Zigmond & Snaith, 1983), Global Anxiety Visual Analog Scale (GA-VAS) (Williams et al., 2010), State-Trait Anxiety Inventory (STAI) (Spielberger, 2010), Profile of Mood States (POMS 2-A) (Searight, 2017)), Local-Global Task (Navon, 1977), Stroop Task (Stroop, 1935), Toronto Mindfulness Scale (TMS) (Lau et al., 2006). Phenomenological Consciousness Inventory (PhCI) (Pekala et al., 1991).

(10) Data Analysis

Data analysis will be multi-pronged:
1. Determine group-effect on the behavioral metrics using an ANOVA, correcting for multiple comparisons across behavioral metrics.
2. Conduct univariate and multivariate machine learning analyses to determine EEG derivatives informative of subject group. This includes, but is not limited to classifying subjects by their group according to: Brain Integration Score, Intra Hemispheric Coherence, Frontal Asymmetry, Imaginary Coherence, Entropy Measures, Lempel-Ziv Complexity, spectral power analysis, and dynamic connectivity across functional networks.
3. Account for individual differences in EEG response and impact on mood states as a function of other behavioral measures collected (e.g. Openness to experience)
(11) Composition and Control Software

The inventors' proprietary plugin gives the content creator the ability to create dynamic light patterns in various LED colors. The effects generated can be recorded into a track or played in real-time on the device. The plugin environment allows synchronization between the different light patterns and audio tracks that constitute a full experience.

The content creator defines the time-varying light pattern by mapping the amplitude of generated waveforms to the brightness of the LEDs of a corresponding channel The plugin instantiates brightness “oscillators,” which in turn generate waveforms based on the following user-controlled oscillation parameters:
Waveform Shape: Square wave (100% brightness, 0% brightness), Sinewave, Sawtooth wave with a falling edge, Sawtooth wave with a rising edge.
Frequency: the frequency of the waveform
Phase: the phase shift of a specific waveform (0-180 degrees)
Depth: Scaling of the base of the wave. The waveform will oscillate between 100% brightness and the depth percentage value, where 0% corresponds to 0% brightness.
Width: the width of the square wave as a percentage of the period (can be thought of as a PWM scaling). It only applies to square waves.
Master Brightness: Limit the height of the waveform as a percentage of the maximum current output of the LED controller

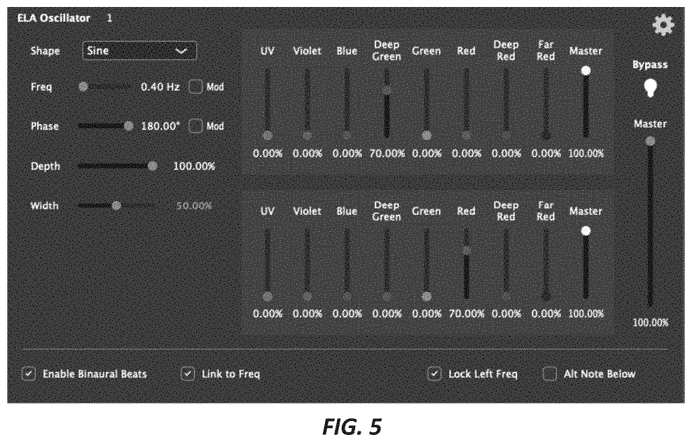

Each oscillator has 2 color palettes, which are user-controlled using 8 color sliders (one for each LED color channel), as illustrated in FIG. 5.

The waveform modulation controller allows a content creator to create dynamic time-varying light patterns between 2 palettes easily.

For example, a user may set up an initial green oscillator outputting a sine wave at 5 Hz and 0 degrees phase shift. A second oscillator is added, outputting the same waveform on the red channel but with a 180-degree phase shift. When played back, the user will have created an alternating red/green Pulse. This is a simple example; An unlimited number of oscillators can be simultaneously instantiated, allowing for much more complicated sequential pulsing effects.

Individual color channel brightness values are capped at a fixed saturation value—if multiple oscillators sum a specific channel beyond 100%, the brightness value is clipped.

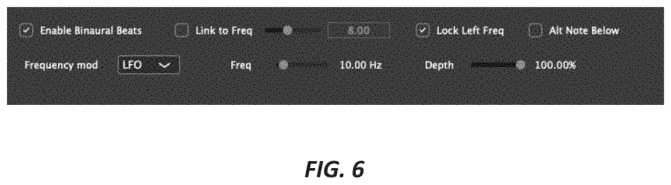

As illustrated in FIG. 6, the Oscillators' audio output allows the user to create synchronous and a-synchronous binaural beats with the Light patterns. Midi control triggers the generator at a pitch designated by an external controller.
Link to Frequency: Synchronize to master waveform oscillator
Lock Left Frequency: Hz output of left channel freq locked at pitch designated by external midi.
Alt Note Below: Right channel Hz is variable at values + and − the left freq or limited to minus values only

(12) Study Design

### Potential Behavioral Metrics

| | What Does It | | | | |
|---|---|---|---|---|---|
| Scale Name | Measure? | Length | | Comments | Reference |
| Hospital Anxiety | Asses generalized | 7 items < | | Has excellent | Zigmond & |
| and Depression | anxiety including | 5 minutes | | reliability and good | Snaith (1983) |
| Scale - Anxiety | tension, worry, | | | validity, and is also | |
| (HADS - Anxiety) | fear, panic, | | | sensitive to change. | |
| | difficulties in | | | | |
| | relaxing, and | | | | |
| | restlessness. | | | | |
| Global Anxiety | Measures very | 5 | seconds | Allows for free- | Williams et al. |
| Visual Analog | quickly of not at | | | form marking on | (2010) |
| Scale (GA-VAS) | all anxious <--> | | | a line that is then | |
| | extremely | | | measured to the | |
| | anxious | | | nearest millimeter. | |
| State-Trait | Allows for the | 40 items | | The trait measure | Spielberger |
| Anxiety Inventory | capture of an “In | (20 state; | | can be an | (2010) |
| (STAI) | this moment” | 20 trait) | | indicator of test- | |
| | measure as well as | 10 | minutes | retest reliability | |
| | a “general”/trait | | | before and after | |
| | measure. | | | the experience, | |
| | | | | whereas we | |
| | | | | would hope for a | |
| | | | | delta on the state. | |
| | | | | The state anxiety is | |
| | | | | more responsive to | |
| | | | | change than trait | |
| | | | | anxiety (Julian, | |
| | | | | 2014) | |
| Beck Anxiety | A brief measure of | 21 Items | | Has established | Beck et al. |
| Inventory | anxiety with a | 5-10 | minutes | responsiveness to | (1988) |
| | focus on somatic | | | change in | |
| | symptoms and is | | | psychiatric and | |
| | good at | | | medical | |
| | distinguishing | | | populations. | |
| | anxiety vs. | | | | |
| | depression. | | | | |
| Profile of Mood | Ability to capture | 10 | minutes | Would be important | Searight |
| States (POMS 2- | mood changes and | | | for marketing | (2017) |
| A) | improvements; is | | | claims. | |
| | the gold-standard | | | | |
| | in supplements, | | | | |
| | athletics, etc. | | | | |
| Local-Global | Measures | 5 | minutes | Could be a more | Navon (1977) |
| Task | interference from | | | Reaction-Time | |
| | other features | | | based way to | |
| | when asked to | | | capture one's | |
| | identify either | | | behaviorally | |
| | global or local | | | meaningful impact | |
| | features of a | | | of reduced anxiety | |
| | stimulus. | | | that goes beyond | |
| | | | | self- reporting. | |
| Stroop Task | Measures ability | 5 | minutes | Could be a more | Stroop (1935) |
| | to attend to | | | Reaction-Time | |
| | specific elements | | | based way to | |
| | of a task and | | | capture one's | |
| | scales with | | | behaviorally | |
| | anxiety (i. | | | meaningful impact | |
| | e. performance | | | of reduced anxiety | |
| | worsens with | | | that goes beyond | |
| | higher | | | self- reporting. | |
| | anxiety). | | | | |
| Toronto | The TMS scale is | 2 | minutes | Should be | Lau et al. |
| Mindfulness Scale | intended to | | | included following | (2006) |
| (TMS) | measure “state- | | | the experience to | |
| | like” experiences | | | compare to the | |
| | during an | | | meditation/ | |
| | experience, rather | | | breathwork group. | |
| | than “trait-like” | | | | |
| | cognitive | | | | |
| | dispositions that | | | | |
| | might reflect the | | | | |
| | cognitive | | | | |
| | consequences of | | | | |
| | meditative | | | | |
| | practice. | | | | |
| Five Facet | Identifies one's | 7 | minutes | Should be | Baer et al. |
| Mindfulness | “trait- like” | | | included prior to | (2006) |
| Questionnaire | tendency to be | | | the experience. | |
| | more mindful in | | | | |
| | nature. | | | | |
| NEO-FFI | Openness to | 10-15 | minutes | This should be | McCrae et al. |
| (Specifically the | Experience | | | included after | (2010) |
| Opnness to | | | | reviewing the | |
| Experience | | | | SloverLinett | |
| aspect) | | | | responses. It | |
| | | | | seems people | |
| | | | | interested in this | |
| | | | | in the first place | |
| | | | | would benefit | |
| | | | | more. | |
| Phenomenological | Will allow for a | 5 | minutes | | Pekala et al. |
| Consciousness | general | | | | (1991) |
| Inventory (PhCI) | capture/fingerprint | | | | |
| | of one's | | | | |
| | phenomenological | | | | |
| | experience. | | | | |

### Potential Biometrics w/Peripherals

| Measurement | What Does It Tell Us? | Material/When |
|---|---|---|
| Electromyography (EMG) | Detects the electric potential | During |
| | generated by muscle cells | |
| | and will allow for a metric | |
| | of movement throughout the | |
| | experience that can be | |
| | regressed out of the EEG | |
| | dataset to avoid any motion- | |
| | related artifacts. Can also be | |
| | used to compare against | |
| | controls. | |
| Bio-Impedance-Based | Allows for subtle detection | Before (5 min), During, and |
| Respiration Rate | of increases or decreases in | After (5 min) |
| | state-anxiety over the course | |
| | of the experience | |
| Heart Rate Variability | Anxiety is hallmarked by | Before and After |
| (HRV) | reduced resting-state heart | |
| | rate variability. | |
| Oxygen Saturation (SpO2) | Detects oxygen levels in the | Before (5 min), During, and |
| | blood and, as a result, CO2 | After (5 min) |
| | levels, which can be used to | |
| | detect hyperventilation | |
| | during an experience. | |
| Temperature | Basal body temperature + | Before (5 min), During, and |
| | experience-induced changes | After (5 min) |
| | in temperature. Anxiety can | |
| | alter body temperature by 1- | |
| | degree Fahrenheit, so it | |
| | stands to reason an | |
| | anxiolytic could “cool” an | |
| | individual as well. | |
| Galvanic Skin Response | Measures electrodermal | Before (5 min), During, and |
| (GSR) | activity. Skin resistance | After (5 min) |
| | varies with the state of | |
| | sweat glands in the skin-- an | |
| | indicator of sympathetic | |
| | nervous system arousal. | |
| Saliva Amylase Activity | sAA concentrations increase | Before and After |
| (sAA) | as a function of anxiety. | |
| | Provides a strong biological | |
| | marker | |

### Potential Neuroscientific Metrics

| Measurement | What Does It Measure? | Material/When |
|---|---|---|
| Brain Integration Score | Frontal coherence, a | EEG - Before vs. After |
| (BIS) | measure of coordinated | |
| | functioning of executive | |
| | brain areas; | |
| | Higher alpha and lower | |
| | gamma EEG, a change in | |
| | processing style from | |
| | attention to outer | |
| | boundaries (gamma EEG) | |
| | to attention to one's inner | |
| | state of well- being (alpha | |
| | EEG); | |
| | More appropriate cortical | |
| | preparatory response, a | |
| | measure of efficiency of | |
| | applying mental and motor | |
| | resources to the task. | |
| Inter and Intra Hemispheric | Shows changes in neuro- | Before vs. After |
| Coherence | psycho-physiological | |
| | functioning that are not | |
| | detectable by simply | |
| | measuring amplitude or | |
| | power spectra. Can show | |
| | abnormalities in patients | |
| | with anxiety. | |
| Frontal Assymetry | The difference in mean | Before vs. After |
| | alpha band power between | |
| | the left and right frontal | |
| | cortex. | |
| | Greater left frontal activity | |
| | should translate to less | |
| | anxiety. | |
| Microstate Analysis | | |
| Complexity Analysis | | |
| Coherence | | |
| Imaginary Coherence | Can be used as an | |
| | alternative method towards | |
| | estimating functional | |
| | connectivity across | |
| | channels/regions. | |
| Entropy Measures (Spectral | | |
| Entropy, Symbolic Transfer | | |
| Entropy, Cross- | | |
| Approximate Entropy, | | |
| Permutation Entropy, | | |
| Weighted symbolic mutual | | |
| information | | |
| Bispectral Index | | |
| Predominant Background | | |
| Activity | | |
| Wavelet Decomposition | Should be used for pre- | |
| | processing that creates a | |
| | more sensitive set of | |
| | features for analysis. | |
| Intrinsic Network Reactivity | | |
| Index | | |
| Debiased Weighted Phase | Can be used as an | |
| Lag Index | alternative method towards | |
| | estimating functional | |
| | connectivity across | |
| | channels/regions. | |
| Lempel-Ziv Complexity | | |
| Dynamic Connectivity | The co-fluctuation of | Before, During, After - Will |
| Across Functional Networks | neural activity across | be specifically useful in |
| (Band by Band) | different nodes in the | determining if specific |
| | brain belonging to | functional networks change |
| | specific functional brain | their activity as a function of |
| | networks, whose roles | the experience. The Before |
| | and functions are well | data will also be used to |
| | defined. | predict individual differences |
| | This analysis will allow for | to identify whose brain is |
| | us to determine network- | positioned to benefit the most |
| | specific modulations and if | from the experience. The |
| | that lines up with the | Before vs. After contrast will |
| | literature (e.g. decreased | show if there were any |
| | activity in the Default | functional changes within the |
| | Mode Network during | networks (and within any |
| | times of deep relaxation). | specific frequency band) as a |
| | | function of the experience. |
| | | We will also see if this |
| | | change is observable during |
| | | the experience as well. |
| Spectral Analysis | Allows for a | Before, During, After - Will |
| | determination of which | be specifically used to look |
| | frequency bands are | at periods of “entrainment” |
| | changing as a function of | to see if there is an increase |
| | the experience and during | in frequency during the |
| | specific epochs of the | presentation of auditory |
| | experience. | sounds within that same |
| | | frequency band. |
| Spectral Power Analysis | Allows for a | Before, During, After - Will |
| | determination of which | be specifically used to look |
| | frequency bands are | at periods of “entrainment” |
| | changing as a function of | to see if there is an increase |
| | the experience and during | in frequency band power |
| | specific epochs of the | during the presentation of |
| | experience. | auditory sounds within that |
| | | same frequency band. |

(13) Subject Groups

The study will be placebo-controlled and double-blind.

Participants will be randomly assigned to either the control or experimental group using an auto-randomization procedure, but balanced for Male and Female within each group. Control groups will be age-matched to the experimental groups.

Use of language herein such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” are intended to be inclusive of both a single item (just X, or just Y, or just Z) and multiple items (i.e., {X and Y}, {X and Z}, {Y and Z}, or {X, Y, and Z}). “At least one of” is not intended to convey a requirement that each possible item must be present.

Further aspects of the present disclosure are provided by the subject matter of the following clauses.

A method of treating a medical indication, comprising: administering a dosage of light to a patient.

The method according to the preceding clause, wherein the dosage of light is administered to the patient's eyes.

The method according to any preceding clause, wherein the dosage of light is administered to the patient's eyes while the patient's eyes are closed.

The method according to any preceding clause, wherein the dosage of light is defined according to one or more predetermined light wavelengths.

The method according to any preceding clause, wherein the dosage of light is defined according to one or more predetermined pulse frequencies.

The method according to any preceding clause, wherein the dosage of light is defined according to one or more predetermined areas within the patient's field of vision.

The method according to any preceding clause, wherein the one or more medical indications includes pain, headache, or migrane.

The method according to any preceding clause, wherein the one or more medical indications includes hypertension.

The method according to any preceding clause, wherein the one or more medical indications includes Alzheimer's or dementia.

The method according to any preceding clause, wherein the one or more medical indications includes attention-deficit/hyperactivity disorder.

A method of treating one or more medical indications using a device that includes at least one controller and a light emitter module, comprising: controlling the light emitter module to emit light at a first pulse frequency in a direction of a user's eyes; and controlling the light emitter module to change the light emission from the first pulse frequency to a second pulse frequency.

The method according to any preceding clause, wherein the one or more medical indications includes pain, headache, or migrane.

The method according to any preceding clause, wherein the one or more medical indications includes hypertension.

The method according to any preceding clause, wherein the one or more medical indications includes Alzheimer's or dementia.

The method according to any preceding clause, wherein the one or more medical indications includes attention-deficit/hyperactivity disorder.

The method according to any preceding clause, further comprising: controlling an audio emitter to emit sound at a third pulse frequency.

The method according to any preceding clause, wherein the first pulse frequency is equal to the third pulse frequency.

The method according to any preceding clause, wherein the light at the first pulse frequency and the sound at the third pulse frequency are emitted with a phase offset.

The method according to any preceding clause, wherein the controlling of the light emitter module includes emitting the light when the user's eyes are closed.

The method according to any preceding clause, wherein the controlling of the light emitter module includes emitting the light at one or more non-visible wavelengths.

The method according to any preceding clause, wherein the light emitter module includes a plurality of light emitters, each light emitter corresponding to one of a plurality of color channels, wherein the plurality of color channels includes at least eight color channels, wherein the controlling of the light emitter module includes controlling the plurality of color channels.

The method according to any preceding clause, wherein the controlling of the light emitter module includes sweeping the pulse frequency of the light emission from the first pulse frequency to the second pulse frequency.

The method according to any preceding clause, wherein the sweeping of the pulse frequency of the emitted light includes continuously changing the pulse frequency of the light emission from the first pulse frequency to the second pulse frequency.

The method according to any preceding clause, wherein the sweeping of the pulse frequency of the emitted light includes incrementally changing the pulse frequency of the light emission from the first pulse frequency to the second pulse frequency.

A system comprising: at least one controller; and a light emitter module, wherein the light emitter module is configured to administer at least one dosage of light to a patient.

The system according to the preceding clause, wherein the light emitter module is configured to administer at least one dosage of light to the patient's eyes.

The system according to any preceding clause, wherein the light emitter module is configured to administer at least one dosage of light to the patient's eyes while the patient's eyes are closed.

The system according to any preceding clause, wherein the dosage of light is defined according to one or more predetermined light wavelengths.

The system according to any preceding clause, wherein the dosage of light is defined according to one or more predetermined pulse frequencies.

The system according to any preceding clause, wherein the dosage of light is defined according to one or more predetermined areas within the patient's field of vision.

The system according to any preceding clause, wherein the dosage is defined according to a photon quantity,

A method of treating one or more medical indications, comprising: controlling the light emitter module to emit light at a first pulse frequency in a direction of a user's eyes; and controlling the light emitter module to change the light emission from the first pulse frequency to a second pulse frequency.

Although the foregoing description is directed to the embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

We claim:

1. A method of treating a medical indication, comprising:
administering a dosage of light to a patient.

2. The method of claim 1, wherein the dosage of light is administered to the patient's eyes.

3. The method of claim 2, wherein the dosage of light is administered to the patient's eyes while the patient's eyes are closed.

4. The method of claim 1, wherein the dosage of light is defined according to one or more predetermined light wavelengths.

5. The method of claim 1, wherein the dosage of light is defined according to one or more predetermined pulse frequencies.

6. The method of claim 2, wherein the dosage of light is defined according to one or more predetermined areas within the patient's field of vision.

7. The method of claim 1, wherein the one or more medical indications includes pain, headache, or migrane.

8. The method of claim 1, wherein the one or more medical indications includes hypertension.

9. The method of claim 1, wherein the one or more medical indications includes Alzheimer's or dementia.

10. The method of claim 1, wherein the one or more medical indications includes attention-deficit/hyperactivity disorder.

11. A method of treating one or more medical indications using a device that includes at least one controller and a light emitter module, comprising:
controlling the light emitter module to emit light at a first pulse frequency in a direction of a user's eyes; and
controlling the light emitter module to change the light emission from the first pulse frequency to a second pulse frequency.

12. The method of claim 11, wherein the one or more medical indications includes pain, headache, or migrane.

13. The method of claim 11, wherein the one or more medical indications includes hypertension.

14. The method of claim 11, wherein the one or more medical indications includes Alzheimer's or dementia.

15. The method of claim 11, wherein the one or more medical indications includes attention-deficit/hyperactivity disorder.

16. The method of claim 11, further comprising:
controlling an audio emitter to emit sound at a third pulse frequency.

17. The method of claim 16, wherein the first pulse frequency is equal to the third pulse frequency.

18. The method of claim 17, wherein the light at the first pulse frequency and the sound at the third pulse frequency are emitted with a phase offset.

19. The method of claim 11, wherein the controlling of the light emitter module includes emitting the light when the user's eyes are closed.

20. The method of claim 11, wherein the controlling of the light emitter module includes emitting the light at one or more non-visible wavelengths.

21. The method of claim 11, wherein the light emitter module includes a plurality of light emitters, each light emitter corresponding to one of a plurality of color channels,
wherein the plurality of color channels includes at least eight color channels,
wherein the controlling of the light emitter module includes controlling the plurality of color channels.

22. The method of claim 11, wherein the controlling of the light emitter module includes sweeping the pulse frequency of the light emission from the first pulse frequency to the second pulse frequency.

23. The method of claim 22, wherein the sweeping of the pulse frequency of the emitted light includes continuously changing the pulse frequency of the light emission from the first pulse frequency to the second pulse frequency.

24. The method of claim 22, wherein the sweeping of the pulse frequency of the emitted light includes incrementally changing the pulse frequency of the light emission from the first pulse frequency to the second pulse frequency.

25. A system comprising:
at least one controller; and
a light emitter module,
wherein the light emitter module is configured to administer at least one dosage of light to a patient.

26. The system of claim 25, wherein the light emitter module is configured to administer at least one dosage of light to the patient's eyes.

27. The system of claim 26, wherein the light emitter module is configured to administer at least one dosage of light to the patient's eyes while the patient's eyes are closed.

28. The system of claim 25, wherein the dosage of light is defined according to one or more predetermined light wavelengths.

29. The system of claim 25, wherein the dosage of light is defined according to one or more predetermined pulse frequencies.

30. The system of claim 26, wherein the dosage of light is defined according to one or more predetermined areas within the patient's field of vision.

31. The system of claim 25, wherein the dosage is defined according to a photon quantity,

32. A method of treating one or more medical indications, comprising:
controlling the light emitter module to emit light at a first pulse frequency in a direction of a user's eyes; and
controlling the light emitter module to change the light emission from the first pulse frequency to a second pulse frequency.
